(19) United States
(12) Patent Application Publication
(10) Pub. No.: US 2005/0267354 A1
(43) Pub. Date: Dec. 1, 2005

(54) System and method for providing computer assistance with spinal fixation procedures

(76) Inventors: Joel Marquart, Davie, FL (US); Louis K. Arata, Mentor, OH (US); Randall Hand, Pembroke Pines, FL (US); Arthur E. Quaid III, Hollywood, FL (US); Rony A. Abovitz, Hollywood, FL (US); Richard Dickerson, Rockaway, NJ (US)

Correspondence Address: MUNSCH, HARDT, KOPF & HARR, P.C., INTELLECTUAL PROPERTY DOCKET CLERK, 1445 ROSS AVENUE, SUITE 4000, DALLAS, TX, 75202-2790, US

(21) Appl. No.: 11/006,503

(22) Filed: Dec. 6, 2004

Related U.S. Application Data
(63) Continuation of application No. 10/771,850, filed on Feb. 4, 2004.
(60) Provisional application No. 60/444,975, filed on Feb. 4, 2003.
(60) Provisional application No. 60/444,824, filed on Feb. 4, 2003.
(60) Provisional application No. 60/445,078, filed on Feb. 4, 2003.
(60) Provisional application No. 60/444,989, filed on Feb. 4, 2003.
(60) Provisional application No. 60/444,988, filed on Feb. 4, 2003.
(60) Provisional application No. 60/445,002, filed on Feb. 4, 2003.
(60) Provisional application No. 60/445,001, filed on Feb. 4, 2003.
(60) Provisional application No. 60/319,924, filed on Feb. 4, 2003.

(51) Int. Cl. A61B 5/05

(52) U.S. Cl. 600/411; 600/407

(57) ABSTRACT

A system and method for providing computer assistance for performing a medical procedure, for example spinal fixation, provides a graphical user interface to guide and/or assist a user, for example a surgeon, performing the medical procedure, whether surgical or non-surgical. The computer-assisted system comprises a software application, for example a spinal fixation application, that may be used for a medical procedure, for example spine linking, etc.

This patent application is a continuation of U.S. patent application Ser. No. 10/771,850, entitled “System and Method for Providing Computer Assistance with Spinal Fixation Procedures,” filed Feb. 4, 2004; and claims the benefit of U.S. provisional patent application Ser. No. 60/444,975, entitled “System and Method for Providing Computer Assistance with Spinal Fixation Procedures,” the disclosure of which is incorporated herein by reference. This application relates to the following United States provisional patent applications: Ser. No. 60.444,824, entitled “Interactive Computer-Assisted Surgery System and Method”; Ser. No. 60/445,078, entitled “Computer-Assisted Knee Replacement Apparatus and Method”; Ser. No. 60/444,989, entitled “Computer-Assisted External Fixation Apparatus and Method”; Ser. No. 60/444,988, entitled “Computer-Assisted Knee Replacement Apparatus and Method”; Ser. No. 60/445,002, entitled “Method and Apparatus for Computer Assistance With Total Hip Replacement Procedure”; Ser. No. 60/445,001, entitled “Method and Apparatus for Computer Assistance With Intramedullary Nail Procedure”; and Ser. No. 60/319,924, entitled “Portable, Low-Profile Integrated Computer, Screen and Keyboard for Computer Surgery Applications”; each of which was filed on Feb. 4, 2003 and is incorporated herein by reference. This application also relates to the following applications: U.S. patent application Ser. No. 10/772,083, entitled “Interactive Computer-Assisted Surgery System and Method”; U.S. patent application Ser. No. 10/772,139, entitled “Computer-Assisted Knee Replacement Apparatus and Method”; U.S. patent application Ser. No. 10/772,142, entitled Computer-Assisted External Fixation Apparatus and Method”; U.S. patent application Ser. No. 10/772,085, entitled “Computer-Assisted Knee Replacement Apparatus and Method”; U.S. patent application Ser. No. 10/772,092, entitled “Method and Apparatus for Computer Assistance With Total Hip Replacement Procedure”; U.S. patent application Ser. No. 10/771,851, entitled “Method and Apparatus for Computer Assistance With Intramedullary Nail Procedure”; and U.S. patent application Ser. No. 10/772,137, entitled “Portable Low-Profile Integrated Computer, Screen and Keyboard for Computer Surgery Applications”; each of which was filed on Feb. 4, 2004 and is incorporated herein by reference.

### TECHNICAL FIELD OF THE INVENTION

The present invention relates generally to computer-assisted surgery systems and surgical navigation systems.

### BACKGROUND OF THE INVENTION

Image-based surgical navigation systems display the positions of surgical tools with respect to preoperative (prior to surgery) or intraoperative (during surgery) image data sets. Two and three dimensional image data sets are used, as well as time-variant images data (i.e. multiple data sets taken at different times). Types of data sets that are primarily used include two-dimensional fluoroscopic images and three-dimensional data sets include magnetic resonance imaging (MRI) scans, computer tomography (CT) scans, positron emission tomography (PET) scans, and angiographic data. Intraoperative images are typically fluoroscopic, as a C-arm fluoroscope is relatively easily positioned with respect to patient and does not require that a patient be moved. Other types of imaging modalities require extensive patient movement and thus are typically used only for preoperative and post-operative imaging.

The most popular navigation systems make use of a tracking or localizing system to track tools, instruments and patients during surgery. These systems locate in predefined coordinate space specially recognizable markers that are attached or affixed to, or possibly inherently a part of, an object such as an instrument or a patient. Markers can take several forms, including those that can be located using optical (or visual), electromagnetic, radio or acoustic methods. Furthermore, at least in the case of optical or visual systems, location of an object's position may be based on intrinsic features or landmarks that, in effect, function as recognizable markers. Markers will have a known, geometrical arrangement with respect to, typically, an end point and/or axis of the instrument. Thus, objects can be recognized at least in part from the geometry of the markers (assuming that the geometry is unique), and the orientation of the axis and location of endpoint within a frame of reference deduced from the positions of the markers.

Present day tracking systems are typically optical, functioning primarily in the infrared range. They usually include a stationary stereo camera pair that is focused around the area of interest and sensitive to infrared radiation. Markers emit infrared radiation, either actively or passively. An example of an active marker is a light emitting diodes (LEDs). An example of a passive marker is a reflective marker, such as ball-shaped marker with a surface that reflects incident infrared radiation. Passive systems require a an infrared radiation source to illuminate the area of focus. A magnetic system may have a stationary field generator that emits a magnetic field that is sensed by small coils integrated into the tracked tools.

Most CAS systems are capable of continuously tracking, in effect, the position of tools (sometimes also called instruments). With knowledge of the position of the relationship between the tool and the patient and the patient and an image data sets, a system is able to continually superimpose a representation of the tool on the image in the same relationship to the anatomy in the image as the relationship of the actual tool to the patient's anatomy. To obtain these relationships, the coordinate system of the image data set must be registered to the relevant anatomy of the actual patient portions of the of the patient's anatomy in the coordinate system of the tracking system. There are several known registration methods.

In CAS systems that are capable of using two-dimensional image data sets, multiple images are usually taken from different angles and registered to each other so that a representation of the tool or other object (which can be real or virtual) can be, in effect, projected into each image. As the position of the object changes in three dimensional space, its projection into each image is simultaneously updated. In order to register two or more two-dimensional data images together, the images are acquired with what is called a registration phantom in the field of view of the image device. In the case of a two dimensional fluoroscopic images, the phantom is a radio-translucent body holding radio-opaque fiducials having a known geometric relationship. Knowing the actual position of the fiducials in three dimensional space when each of the images are taken permits determination of a relationship between the position of the fiducials and their respective shadows in each of the images. This relationship can then be used to create a transform for mapping between points in three-dimensional space and each of the images. By knowing the positions of the fiducials with respect to the tracking system's frame of reference, the relative positions of tracked tools with respect to the patient's anatomy can be accurately indicated in each of the images, presuming the patient does not move after the image is acquired, or that the relevant are portions of the patient's anatomy is are tracked. A more detailed explanation of registration of fluoroscopic images and coordination of representations of objects in patient space superimposed in the images is found in U.S. Pat. No. 6,198,794 of Peshkin, et al., entitled “Apparatus and method for planning a stereotactic surgical procedure using coordinated fluoroscopy”.

### SUMMARY OF THE INVENTION

A system and method for providing computer assistance for performing a medical procedure, for example spinal fixation, provides a graphical user interface to guide and/or assist a user, for example a surgeon, performing the medical procedure, whether surgical or non-surgical. The computer-assisted system comprises a software application, for example a spinal fixation application, that may be used for a medical procedure, for example spine linking, etc.

The invention is generally directed to improved computer-implemented methods and apparatus for further reducing the invasiveness of surgical procedures, eliminating or reducing the need external fixtures in certain surgical procedures, and/or improving the precision and/or consistency of surgical procedures. The invention finds particular advantage in orthopedic procedures involving implantation of devices, though it may also be used in connection with other types of surgical procedures.

Other aspects and features of the invention will become apparent to those ordinarily skilled in the art upon review of the following description of specific embodiments of the invention in conjunction with the accompanying figures.

### BRIEF DESCRIPTION OF THE DRAWINGS

For a more complete understanding of the present invention, the objects and advantages thereof, reference is now made to the following descriptions taken in connection with the accompanying drawings in which:

FIG. 1 is a block diagram of an exemplary computer-assisted surgery system;

FIG. 2 is a flowchart of a computer-assisted method for spinal fixation;

FIGS. 3A-3K are exemplary screen displays provided during spinal fixation;

FIGS. 4A and 4B are more detailed flowcharts of the method of FIG. 2 for spinal fixation; and

FIG. 5 is a flowchart of a method for linking structure sizing.

### DETAILED DESCRIPTION OF THE DRAWINGS

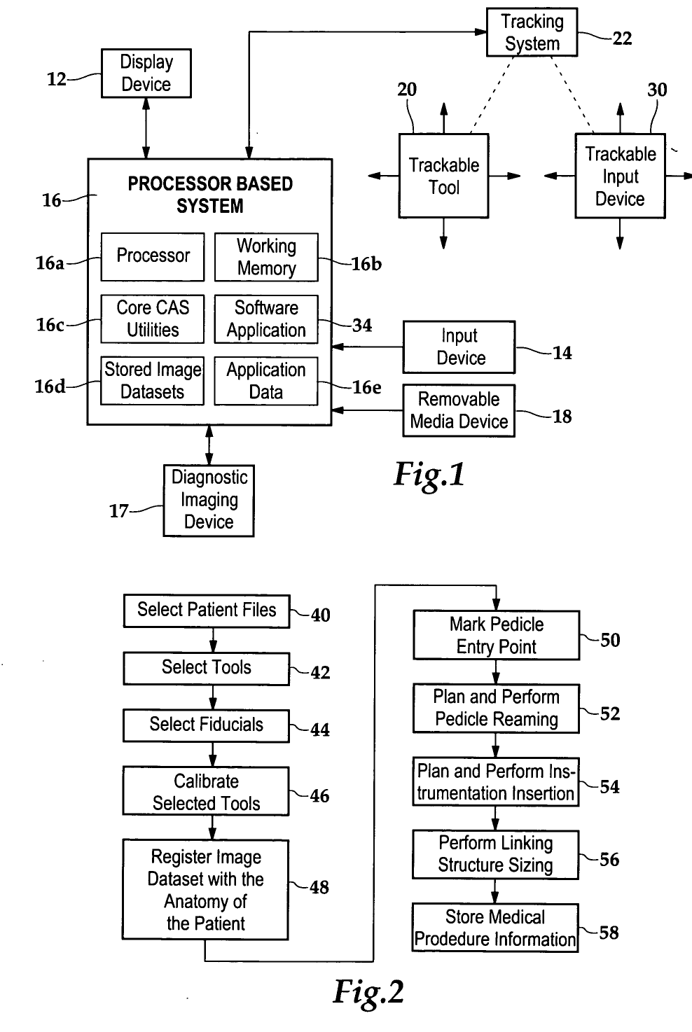

The preferred embodiment of the present invention and its advantages are best understood by referring to FIGS. 1 through 5 of the drawings.

In the following description, like numbers refer to like elements. References to “surgeon” include any user of a computer-assisted surgical system, a surgeon being typically a primary user.

FIG. 1 is a block diagram of an exemplary computer-assisted surgery (CAS) system 10. Computer-assisted surgery system (CAS) 10 comprises a display device 12, an input device 14, and a processor-based system 16, for example a computer. Display device 12 may be any display device now known or later developed for displaying two-dimensional and/or three-dimensional diagnostic images, for example a monitor, a touch screen, a wearable display, a projection display, a head-mounted display, stereoscopic views, a holographic display, a display device capable of displaying image(s) projected from an image projecting device, for example a projector, and/or the like. Input device 14 may be any input device now known or later developed, for example, a keyboard, a mouse, a trackball, a trackable probe and/or the like. The processor-based system is preferably programmable and includes one or more processors 16a, working memory 16b for temporary program and data storage that will be used primarily by the processor, and storage for programs and data, preferably persistent, such as a disk drive. Removable media device 18 can also be used to store programs and/or transferred to or from the transfer programs.

Tracking system 22 continuously determines, or tracks, the position of one or more trackable markers disposed on, incorporated into, or inherently a part of surgical tools or instruments 20 with respect to a three-dimensional coordinate frame of reference. With information from the tracking system on the location of the trackable markers, CAS system 10 is programmed to be able to determine the three-dimensional coordinates of an endpoint or tip of a tool and, optionally, its primary axis using predefined or known (e.g. from calibration) geometrical relationships between trackable markers on the tool and the end point and/or axis of the tool. A patient, or portions of the patient's anatomy, can also be tracked by attachment of arrays of trackable markers.

The CAS system can be used for both planning surgical procedures (including planning during surgery) and for navigation. It is therefore preferably programmed with software for providing basic image guided surgery functions, including those necessary determining the position of the tip and axis of instruments and for registering a patient and preoperative and/or intraoperative diagnostic image data sets to the coordinate system of the tracking system. The programmed instructions for these functions are indicated as core CAS utilities 16c. These capabilities allow the relationship of a tracked instrument to a patient to be displayed and constantly updated in real time by the CAS system overlaying a representation of the tracked instrument on or more graphical images of the patient's internal anatomy on display device 12. The graphical images are constructed from one or more stored image data sets 16d acquired from diagnostic imaging device 17. Imaging device may be a fluoroscope, such as a C-arm fluoroscope, capable of being positioned around a patient laying an operating table. It may also be a MR, CT or other type of imaging device in the room or permanently located elsewhere. Where more than one image is shown, as when multiple fluoroscopic images are simultaneously displayed of display device 12, the representation of the tracked instrument or tool is coordinated between the different images. However, CAS system can be used in some procedures without the diagnostic image data sets, with only the patient being registered. Thus, the CAS system need not support the use of diagnostic images in some applications—i.e. an imageless application.

Furthermore, as disclosed herein, the CAS system may be used to run application-specific programs or software 34 that are directed to assisting a surgeon with planning and/or navigation during specific types of procedures. For example, the software application 34 may display predefined pages or images corresponding to specific steps or stages of a surgical procedure. At a particular stage or part of a program, a surgeon may be automatically prompted to perform certain tasks or to define or enter specific data that will permit, for example, the program to determine and display appropriate placement and alignment of instrumentation or implants or provide feedback to the surgeon. Other pages may be set up to display diagnostic images for navigation and to provide certain data that is calculated by the system for feedback to the surgeon. Instead of or in addition to using visual means, the CAS system could also communicate information in ways, including using audibly (e.g. using voice synthesis) and tactilely, such as by using a haptic interface of device. For example, in addition to indicating visually a trajectory for a drill or saw on the screen, a CAS system may feedback to a surgeon information whether he is nearing some object or is on course with a audible sound or by application of a force or other tactile sensation to the surgeon's hand.

To further reduce the burden on the surgeon, the program may automatically detect the stage of the procedure by recognizing the instrument picked up by a surgeon and move immediately to the part of the program in which that tool is used. Application data 16e—data generated or used by the application—may also be stored processor-based system.

Various types of user input methods can be used to improve ease of use of the CAS system during surgery. One example uses speech recognition to permit a doctor to speak a command. Another example is the use of a tracked object to sense a gesture by a surgeon, which is interpreted as an input to the CAS system. The meaning of the gesture could further depend on the state of the CAS system or the current step in an application process executing on the CAS system. Again, as an example, a gesture may instruct the CAS system to capture the current position of the object. One way of detecting a gesture is to occlude temporarily one or more of the trackable markers on the tracked object (e.g. a probe) for a period of time, causing loss of the CAS system's ability to track the object. A temporary visual occlusion of a certain length (or within a certain range of time), coupled with the tracked object being in the same position before the occlusion and after the occlusion, would be interpreted as an input gesture. A visual or audible indicator that a gesture has been recognized could be used to provide feedback to the surgeon.

If desired, the user may perform a gesture to indicate acceptance of an input provided by the user. When tracking system 22 is a visual tracking system, the gesture comprises visual occlusion of a predetermined portion of trackable tool 20. It is desirable that the occlusion occur for a predetermined occlusion period in order to avoid inadvertent recognition of a gesture.

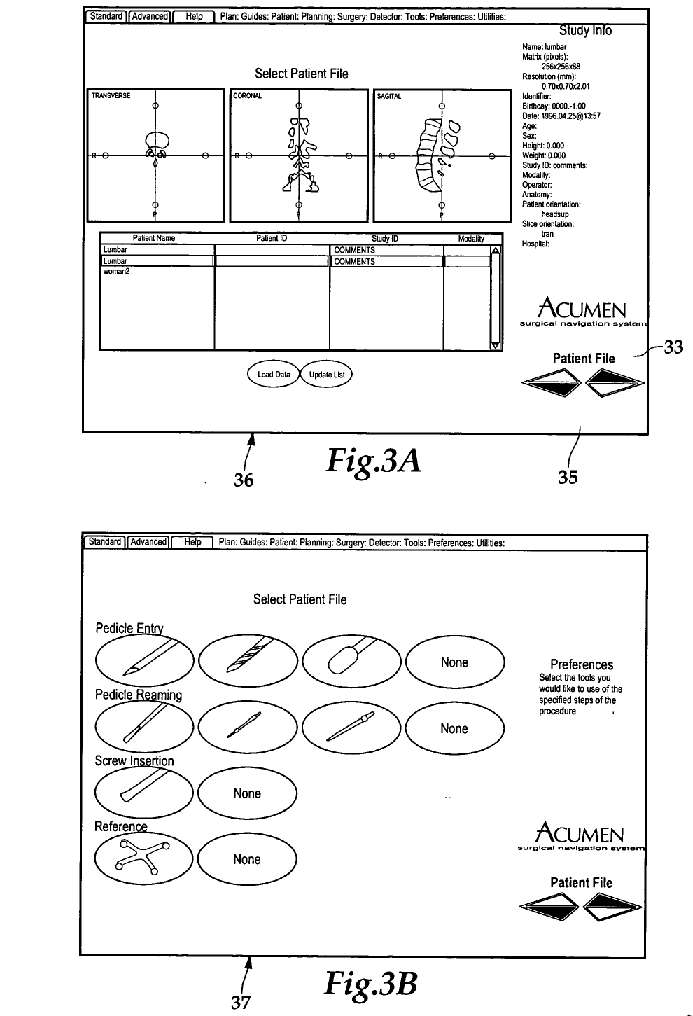

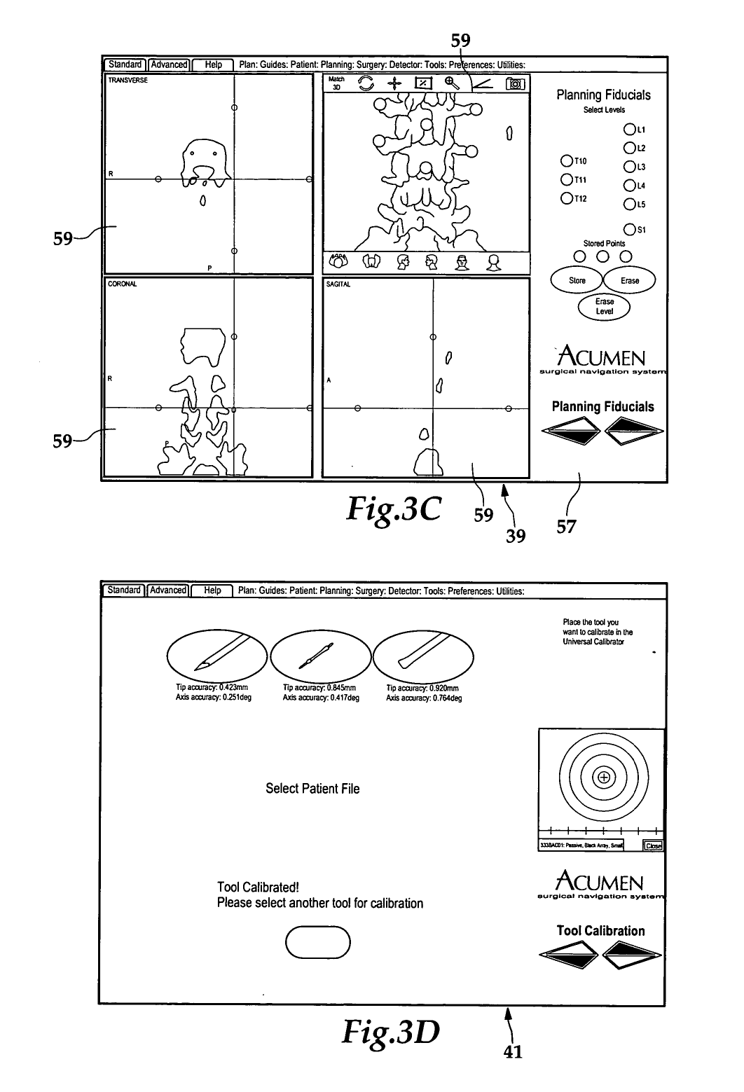

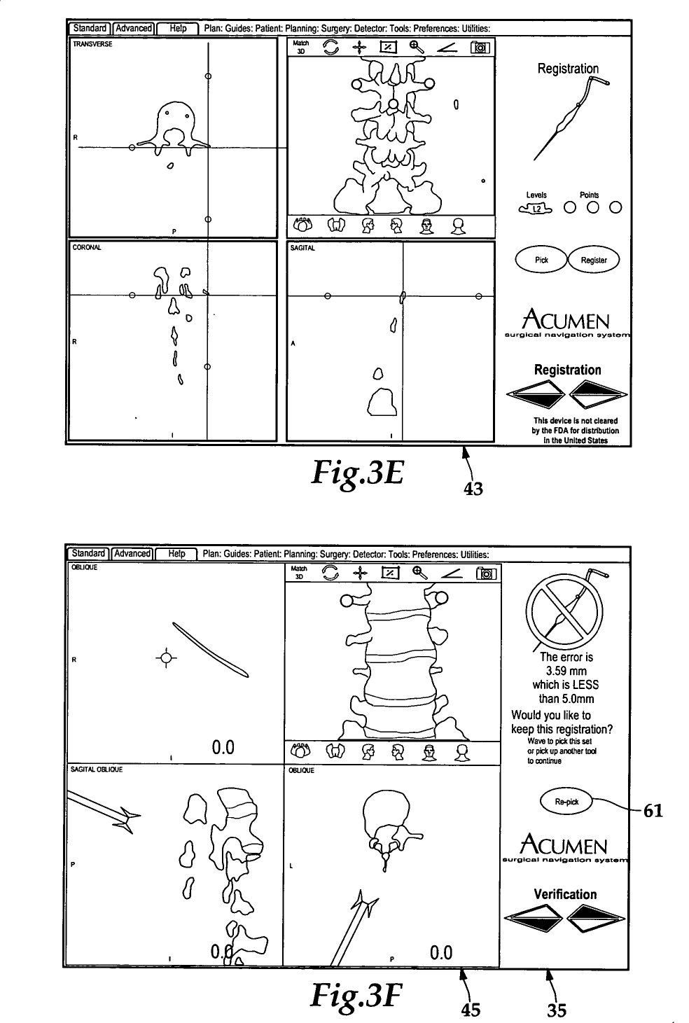

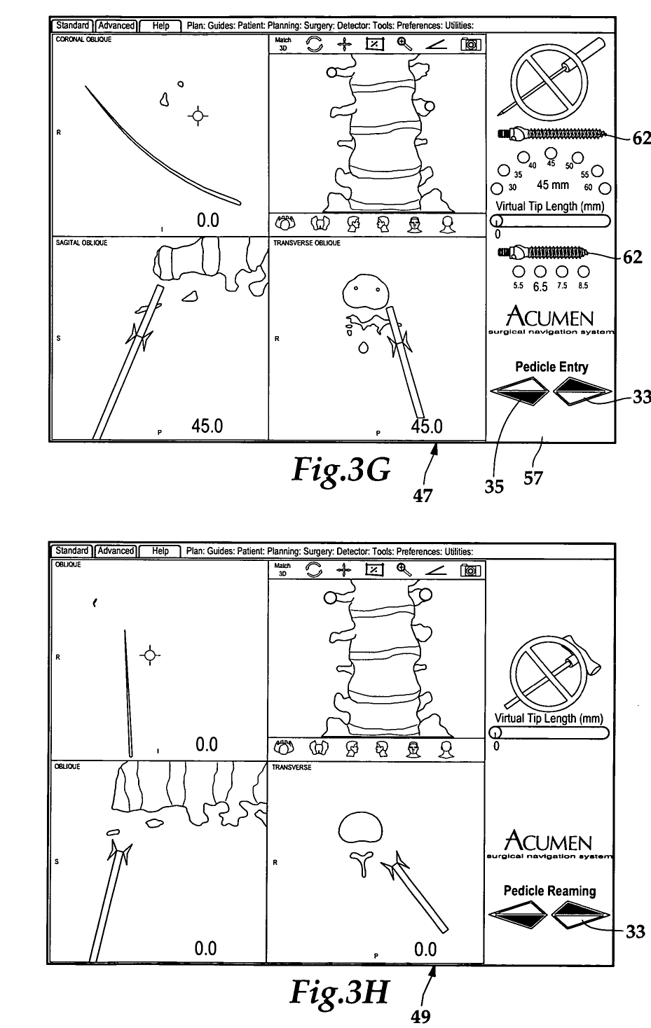

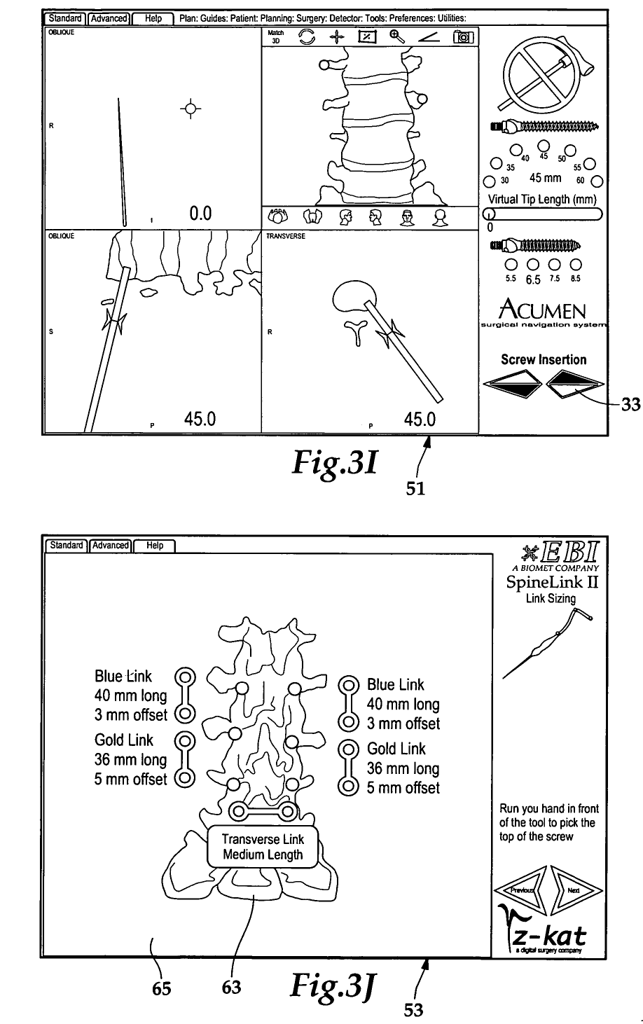

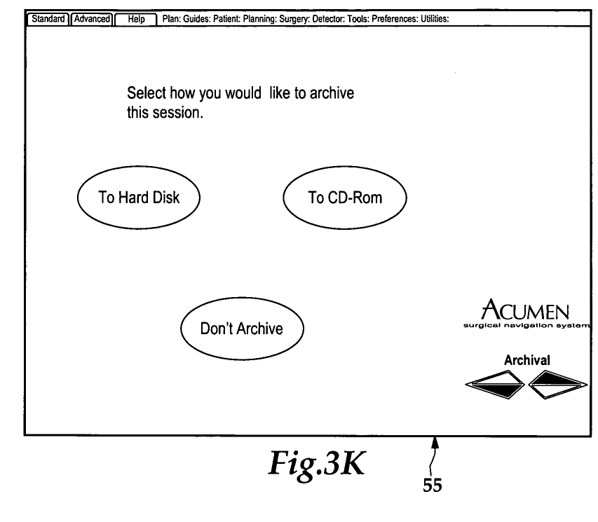

Yet another example of such an input method is the use of tracking system 22 in combination with one or more trackable input devices 30. Defined with respect to the trackable input device 30 are one or more control points, which can be two-dimensional or three-dimensional. These control points are visually indicated on the trackable input device so that a surgeon can see them. For example, the control points may be visually defined on an object by representations of buttons, numbers, letters, words, slides and/or other conventional input devices. The geometric relationship between each control point and trackable input device 30 is known and stored in processor-based system 16. Thus, the processor can determine when another trackable object touches or is in close proximity to a defined control point and recognize it as an indication of a user input to the processor-based systems. For example, when a tip of a tracked pointer is brought into close proximity to one of the defined control points, the processor-based system will recognize the tool near the defined control point and treat it as a user input associated with that defined control point. Preferably, representations on the trackable user input correspond to user input selections (e.g. buttons) on a graphical user interface 36 (FIG. 3A) on display device 12. The trackable input device may be formed on the surface of any type of trackable device, including devices used for other purposes. In a preferred embodiment, representations of user input functions for graphical user interface are visually defined on a rear, flat surface of a base of a tool calibrator. If desired, the trackable input device may be disposable.

Processor-based system 16 is, in one example, a programmable computer that is programmed to execute only when single-use or multiple-use software is loaded from, for example, removable media. The software would include, for example software application 34 for use with a specific type of procedure. Media storing the software application can be sold bundled with disposable instruments specifically intended for the procedure. The application program would be loaded into the processor-based system and stored there for use during one (or a defined number) of procedures before being disabled. Thus, the application program need not be distributed with the CAS system. Furthermore, application programs can be designed to work with specific tools and implants and distributed with those tools and implants. Preferably also the most current core CAS utilities may also be stored with the application program. If the core CAS utilities on the processor-based system are outdated, they can be replaced with the most current utilities.

The method described below utilizes preoperative CT data sets, i.e. CT scans made prior to surgery. Although CT scans offer certain advantages, if desired, image data sets obtained using other two-dimensional or three-dimensional imaging modalities, such as MRI, PET etc. may be used. Furthermore, the image data sets used may be those obtained pre-operatively or intra-operatively. If desired, the image data sets may be time variant, i.e. image data sets taken at different times may be used. Software application 34 may provide visual, auditory or tactile feedback to the user.

FIG. 2 is a flowchart of a computer-assisted method 70 for spinal fixation. Method 70 is intended to provide immobilization and stabilization of spinal segments, as an adjunct to fusion in the treatment of instabilities of thoracic, lumbar, and sacral spine. In general, method 70 comprises one or more of the following steps: selection of patient data (step 40), selection of tools (step 42), selection of fiducials (step 44), calibration of selected tools (step 46), registration of image data sets with the anatomy of the patient (step 48), marking of pedicle entry point (step 50), planning and performance of pedicle reaming (step 52), planning and performance of instrumentation insertion (step 54), performance of linking structure sizing (step 56), storing information about the medical procedure (step 58), etc.

FIGS. 3A-3K are exemplary screen displays provided during spinal fixation. The exemplary screen display 36 of FIG. 3A is provided during selection of patient data (step 40), the exemplary screen display 37 of FIG. 3B is provided during selection of the tools (step 42), the exemplary screen display 39 of FIG. 3C is provided during selection of the fiducials (step 44), the exemplary screen display 41 of FIG. 3D is provided during calibration of the selected tools (step 46), the exemplary screen displays 43 and 45 of FIGS. 3E and 3F respectively are provided during registration of the image data sets with the anatomy of the patient (step 48), the exemplary screen display 47 of FIG. 3G is provided during marking of the pedicle entry point (step 50), the exemplary screen display 49 of FIG. 3H is provided during the planning and performance of pedicle reaming (step 52), the exemplary screen display 51 of FIG. 31 is provided during the planning and performance of instrumentation insertion (step 54), the exemplary screen display 53 of FIG. 3J is provided during the performance of linking structure sizing (step 56), and the exemplary screen display 55 of FIG. 3K is provided during storing of the information about the medical procedure (step 58).

The user may navigate through the different steps of method 70 by selecting a “next screen” icon 33 or a “previous screen” icon 35 on display device 12 to move from one screen to another. The user may navigate through the different steps of method 70 by selecting a “next screen” control point or a “previous screen” control point on trackable input device 30 to move from one screen to another. The user may also proceed to a different step or screen by simply indicating the tool that the user is going to use next. The user may indicate the tool, for example, by simply picking the tool and bringing it into the field of detection of tracking system 22. Software application 34 determines which tool has been selected by the user and automatically displays the screen relevant to the selected tool. For example, if the user picks up a pedicle entry tool, then software application 34 automatically displays the pedicle entry screen (FIG. 3G) on display device 12 so that step 50 of method 70 may be executed.

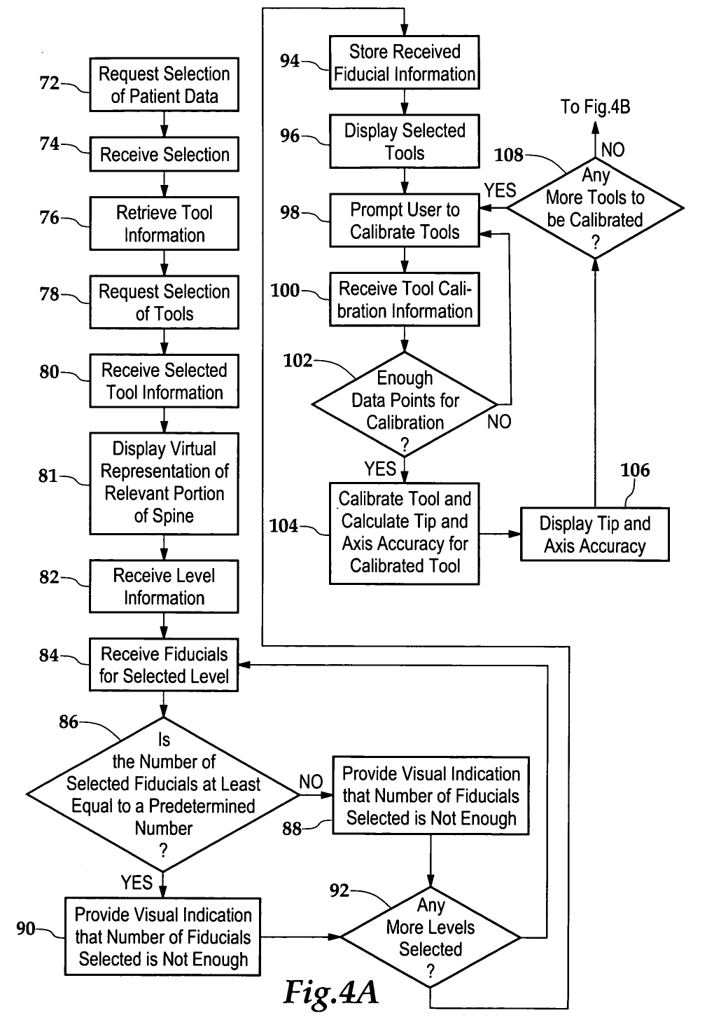

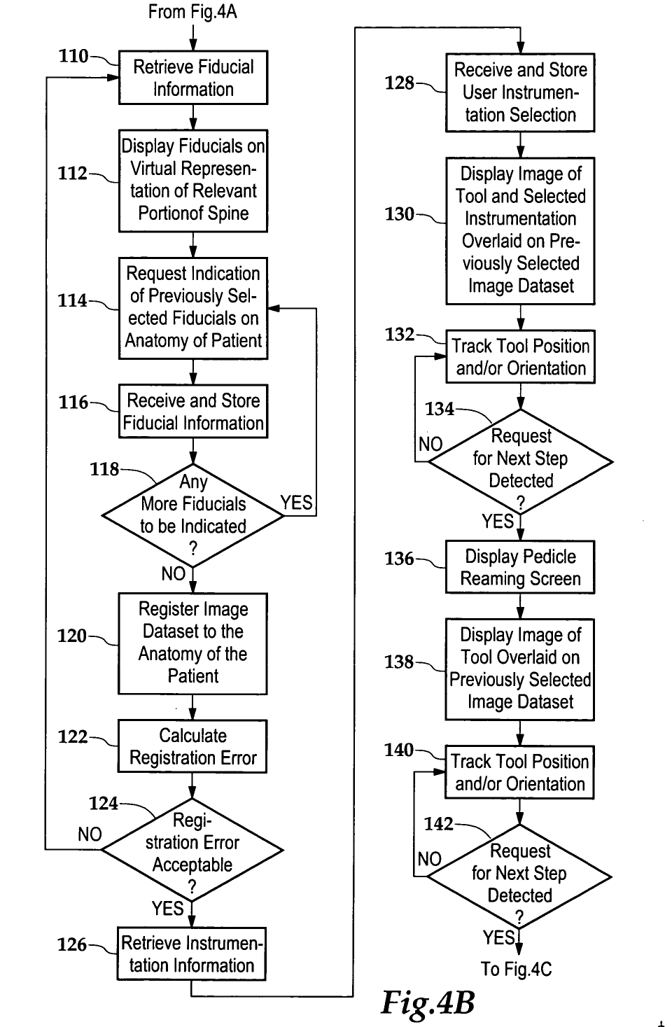

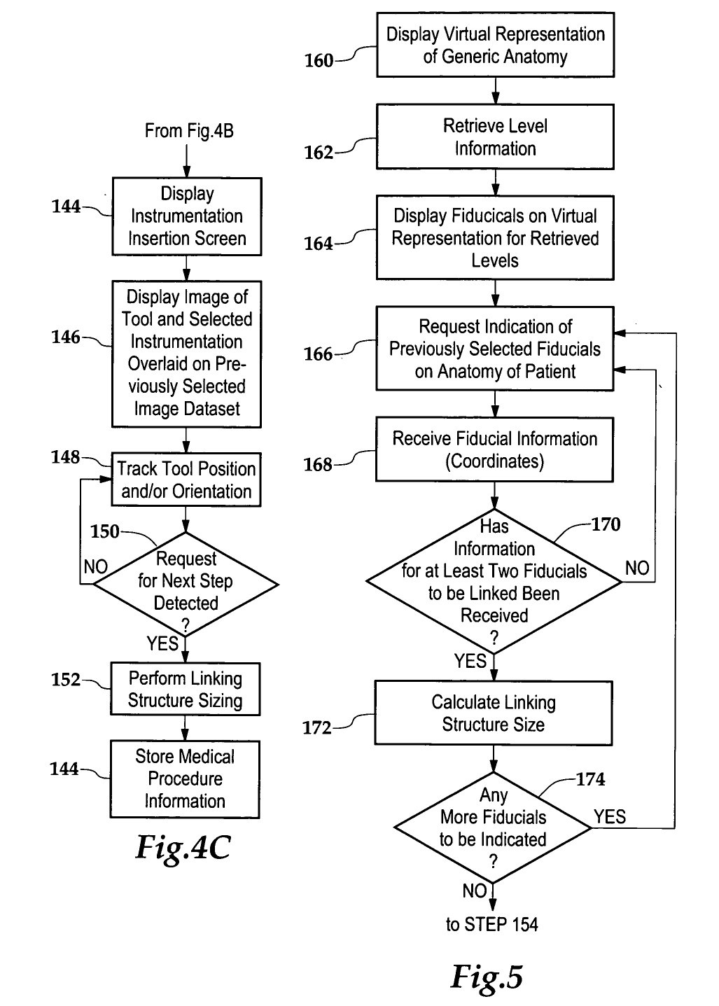

FIGS. 4A and 4B are more detailed flowcharts of method 70 of FIG. 2 for spinal fixation. In step 72, selection of patient data is requested from the user. The patient data may comprise, for example, of image data sets of the relevant portion of the spine of the patient. The image data sets may be two-dimensional or three-dimensional images. Preferably, the image data sets are CT image data sets taken pre-operatively.

The screen display of FIG. 3A may be displayed on display device 12 so that the user may select the patient files to be loaded for the medical procedure. If desired, study information for the displayed image data set may be displayed. In step 74, the selection of the user is received. If desired, the selection may be stored. In step 76, information about the available tools for use during the procedure is retrieved. In step 78, selection of tools is requested. The screen display of FIG. 3B may be displayed on display device 12. As shown in FIG. 3B, the different steps of the medical procedure may be displayed along with representation of the tools available for use during each of the steps. For example, a pedicle instrumentation insertion procedure comprises of three basic steps: 1) Pedicle entry, 2) Pedicle reaming, and 3) Instrumentation insertion, for example screw insertion. So it is desirable that the user select a pedicle entry tool for use during the pedicle entry step (step 50 of FIG. 2), a pedicle reaming tool for use during the pedicle reaming step (step 52) and an instrumentation insertion tool for use during the instrumentation insertion step (step 54). If desired, the user may select an external reference to be used during the procedure.

In step 80, information about the selected tools is received. If desired, the selected tool information may also be stored. In step 81, a virtual representation of the relevant portion of the spine may be displayed. The virtual representation may be a three-dimensional model created based at least in part on the patient data selected in step 72. If desired, the representation may be a generic representation of the relevant portion. FIG. 3C shows an exemplary screen 39 that may be displayed. Screen 39 may comprise of one or more view ports and one or more control windows. In the illustrated embodiment, there are four view ports 59 and one control window 57. Three of the four view ports display transverse, coronal, and sagittal views of the anatomy of interest. These displays are preferably based on the patient information selected in step 72 and may comprise of two-dimensional image data sets. The fourth view port displays a virtual representation of the relevant portion of the spine, which may be used during the medical procedure. The virtual representation may be the three-dimensional model created based at least in part on the patient data selected in step 72.

A plurality of pictorial representations may be provided on one of the view ports, say the fourth view port, to allow the user to change the view. The user may change the views by selecting any one of the pictorial representations by simply clicking on the corresponding pictorial representation. Selecting any of the pictorial representations provides a view of the anatomy from the angle associated with the selected pictorial representation. An advantage of having a pictorial representation is that the user can easily select the desired view.

In step 82, level information about the level of the spine on which the medical procedure is to be performed is received. The control window aids in the selection of the different levels of the spine to be operated on. The available levels of the spine, for example L1, L2, etc. that may be operated on may be displayed in the control window. The user may select a level, for example by selecting one of the levels displayed. After selecting a level the user may select fiducials, for example by selecting points on the virtual representation of the relevant portion of the spine corresponding to the selected level. In step 84, information about fiducials on the selected level is received. In an exemplary embodiment, the received information enables identification of the selected fiducials.

In step 86, a determination is made as to whether the number of fiducials selected for the selected level is at least equal to a predetermined number. In an exemplary embodiment, the predetermined number is three as it is desirable to have at least three fiducials for each level to facilitate registration of the patient image data set to the patient. If the number of selected fiducials is not equal to at least the predetermined number, then in step 88, a visual and/or audio indication may be provided to indicate that the number of fiducials selected for the level is not enough. For example, in the embodiment illustrated, the color of an icon associated with the selected level may be changed to say yellow. Upon seeing the visual indication, the user may decide to provide the minimum number of fiducials desirable or the user may simply ignore the warning.

If the number of selected fiducials is equal to at least the predetermined number, then in step 90, a visual and/or audio indication may be provided to indicate that the number of fiducials selected for the level is enough. For example, in the embodiment illustrated, the color of an icon associated with the selected level may be changed to say green. In step 92, a determination is made as to whether any more levels have been selected. If more levels have been selected then the process starting at step 84 may be executed. Otherwise, in step 94, the received level and/or fiducial information may be stored.

In step 96, representations of the tools selected in step 80 are displayed on display device 12 and in step 98 the user is prompted to calibrate the displayed tools. FIG. 3D shows an exemplary screen 41 that may be displayed. In this step the user may calibrate the tools previously selected. In a spine linking medical procedure the tools are preferably roll calibrated with the aid of trackable input device 30. Any method for tool calibration may be used. The user may calibrate the tools in any order. Software application 34 does not require the user to calibrate the tools in a specified order. It automatically recognizes the tool selected by the user for calibration. In step 100 tool calibration information is received preferably from tracking system 22. In step 102, a determination is made as to whether enough data points for the tool being calibrated have been collected. If enough data points have not been collected, then the process starting at step 98 may be executed.

Otherwise, in step 104, the tool is calibrated. If desired, the tip and axis accuracy for the calibrate tool may be calculated. In step 106, the calculated tip and axis accuracy may be displayed on display device 12. If desired, the tip and axis accuracy may be stored. In step 108, a determination is made as to whether any more of the selected tools are to be calibrated. If there are additional tools to be calibrated, then the process starting at step 98 is executed.

Otherwise, in step 110, fiducial information which was stored in step 94 is retrieved. In step 112, a virtual representation of the relevant portion of the spine may be displayed. The virtual representation may be a three-dimensional model created based at least in part on the patient data selected in step 72. If desired, the representation may be a generic representation of the relevant portion. The retrieved fiducial information is used to highlight the previously selected fiducials on the virtual representation. FIG. 3E shows an exemplary screen 43 that may be displayed.

In step 114, in order to assist the user, the user is prompted to indicate the previously selected fiducials on the spine of the patient. The user may be prompted to indicate the fiducials in any manner now known or later developed. If desired, and as shown in FIG. 3E, the user may be stepped through the fiducial indication process. Information about a previously selected level may be displayed on display device 12. The user may indicate the previously selected fiducials for that level on the spine in any manner now known or later developed, for example by touching the tip of trackable tool 20, such as the probe, to the relevant portions of the spine and providing a signal indicating acceptance of the portion of the spine pointed by the tip of trackable tool 20. In step 116, information about the indicated fiducials is received and stored. In an exemplary embodiment, the received information comprises positional information about the indicated fiducials. If desired, the user may indicate the fiducials in any order. In step 118, a determination is made as to whether there are any more fiducials to be indicated. If there are more fiducials to be indicated, then the process starting at step 114 may be executed.

Otherwise, in step 120, the image data set selected in step 72 is registered to the anatomy of the patient. The registration is performed based at least in part on the fiducial information received in step 118. The image data set may be registered to the anatomy of the patient using any method of registration now known or later developed. In step 122, a registration error is calculated. If desired and as shown in the exemplary screen display of FIG. 3F, the registration error may be displayed on display device 12. If the registration error is more than a predefined threshold, then a visual and/or audio warning may be provided.

In step 124, a determination is made as to whether the registration error is acceptable to the user. The user is given the option to re-register the image data set with the anatomy of the patient. The user may indicate that the registration error is acceptable by either selecting the next screen icon or by simply picking up a tool to be used in a subsequent step. The user may indicate that the registration error is not acceptable by either selecting a “re-pick” icon 61 or by selecting a previous screen icon 35. If the registration error is not acceptable to the user, then the process starting at step 110 may be executed.

Otherwise, in step 126, instrumentation information is retrieved. The instrumentation information may be retrieved from one or more databases or data files. If desired and as shown in the exemplary screen display 47 of FIG. 3G, the retrieved instrumentation information may be displayed on display device 12. The instrumentation 62 may be any type of instrumentation, for example an implant, such as a screw, to be inserted in the spine. The types of instrumentation available for insertion may be displayed, for example in the control window 57, and the user allowed to select the instrumentation he desires to use. The instrumentation may be available in different dimensions. The information that is displayed may be the available dimensions for the instrumentation, for example the available lengths, the available widths, etc.

In step 128, information about the instrumentation selected by the user is received and stored. In step 130, an image of tracked tool 20 with the selected instrumentation extending from the tool is displayed on display device 12 overlaid on the previously selected image data set. The image of the instrumentation reflects the selected dimensions for the instrumentation so that the user can get a better idea of which instrumentation to use and where to select an entry point for the instrumentation.

In step 132, the position and/or orientation of tracked tool 20 with the instrumentation attached to it is tracked and displayed on display device 12 as tracked tool 20 is moved. With the aid of the visual representation, the user can plan the entry point for the instrumentation.

In step 134, a determination is made as to whether a request for the next step has been detected. A determination that the next step (or any other step) has been requested may be made by any method now known or later developed, for example, upon detection of selection of the “next screen” icon 33 on display device 12, upon detection of activation of the “next screen” control point on trackable input device 30, or upon detection of selection, by the user, of a trackable tool that may be associated with a different operation of the pedicle instrumentation insertion procedure. If a request for the next step has not been detected, then the process starting at step 132 may be executed.

If a request for the next step has been detected, then in step 136, a screen 49 (FIG. 3H) for pedicle reaming is displayed. The pedicle reaming screen 49 aids the user in planning the reaming of the bone for insertion of the instrumentation. If desired, the user may specify a virtual tip length, for example by using a slide bar. If the virtual tip length is not zero then the oblique view of the relevant portion of the anatomy is automatically modified. In step 138, an image of tracked tool 20 with a virtual tip equal in length to the selected virtual tip length extending from the end of tracked tool 20 is displayed overlaid on the previously selected image data set. By selecting a virtual tip length equal to the instrumentation length selected in step 128, the user can get a better idea of how deep the inside of the anatomy should be reamed and the path or trajectory for reaming. If desired, a virtual tip length equal to the length of the instrumentation selected in step 128 may be provided by default.

In step 140, the position and/or orientation of tracked tool 20 with the virtual tip (if any) extending from the end of the tool is tracked and displayed on display device 12 as tracked tool 20 is moved. With the aid of the visual representation, the user can plan the path for reaming.

In step 142, a determination is made as to whether a request for the next step has been detected. A determination that the next step (or any other step) has been requested may be made by any method now known or later developed, for example, upon detection of selection of the “next screen” icon 33 on display device 12, upon detection of activation of the “next screen” control point on trackable input device 30, or upon detection of selection, by the user, of a trackable tool that may be associated with a different operation of the pedicle instrumentation insertion procedure. If a request for the next step has not been detected, then the process starting at step 140 may be executed.

If a request for the next step has been detected, then in step 144, a screen 51 (FIG. 31) for instrumentation insertion may be displayed. The instrumentation insertion screen 51 aids the user in planning the insertion of the instrumentation into the spine. The instrumentation insertion screen also provides the user another opportunity to change the instrumentation selected in step 128.

In step 146, an image of tracked tool 20 with the selected instrumentation attached to the tool is displayed overlaid on the previously selected image data set. In step 148, the position and/or orientation of tracked tool 20 with the instrumentation attached to it is tracked and displayed on display device 12 as tracked tool 20 is moved. With the aid of the visual representation, the user can plan the path or trajectory for insertion of the instrumentation.

In step 150, a determination is made as to whether a request for the next step has been detected. A determination that the next step (or any other step) has been requested may be made by any method now known or later developed, for example, upon detection of selection of the “next screen” icon 33 on display device 12, upon detection of activation of the “next screen” control point on trackable input device 30, or upon detection of selection, by the user, of a trackable tool that may be associated with a different operation of the pedicle instrumentation insertion procedure. If a request for the next step has not been detected, then the process starting at step 148 may be executed.

If a request for the next step has been detected, then in step 144, a screen (FIG. 3I) for instrumentation insertion may be displayed. The instrumentation insertion screen 51 aids the user in planning the insertion of the instrumentation into the spine. The instrumentation insertion screen 51 also provides the user another opportunity to change the instrumentation selected in step 128.

If a request for the next step has been detected, then in step 152, a linking structure sizing operation is performed. The process of linking structure sizing is discussed in greater detail herein with reference to the flowchart of FIG. 5. In step 154, the user may be prompted to select how he would like to store information on the medical procedure. In an exemplary embodiment and as shown in the exemplary screen 55 of FIG. 3K, the user is provided the choice of whether or not to store the information. Depending on the selection made by the user the information may be stored, for example on a removable storage media or on a hard drive.

FIG. 5 is a flowchart of a method 152 for linking structure sizing. Linking of portions of the spine facilitates fusion of the portions. It is desirable that the linking structure used be of correct dimensions so that linking may be achieved with less effort and also to facilitate fusion.

In step 160, a virtual representation 63 of the relevant portion of the spine is displayed. The virtual representation may be a three-dimensional model. The three-dimensional model may be created based at least in part on the patient data selected in step 72. If desired, the representation may be a generic representation of the relevant portion. In step 162, information identifying the levels on which the procedure was performed is retrieved. In step 164, the fiducials for the retrieved levels are displayed on the generic representation of the anatomy. The fiducials may be displayed, for example by highlighting the fiducials.

In step 166, in order to assist the user, the user is prompted to indicate the previously selected fiducials or points on the spine anatomy of the patient that are to be linked. The user may be prompted to indicate the fiducials in any manner now known or later developed. If desired, and as shown in FIG. 3J, the user may be stepped through the fiducial indication process. In order to assist the user, the fiducial to be indicated may be highlighted on display device 12. Once the user has indicated the highlighted fiducial on the spine of the patient the process may be repeated for other fiducials. In this manner, the user may be stepped through each previously selected fiducial. If desired, the user may indicate the fiducials in any order. The user may indicate the previously selected fiducials on the spine in any manner now known or later developed, for example by touching the tip of trackable tool 20 to the instrumentation inserted into the relevant portions of the spine and providing a signal indicating acceptance of the portion of the spine pointed by the tip of trackable tool 20.

In step 168, information about the indicated fiducial is received. The received information may be stored, if desired. In an exemplary embodiment, the information received comprises positional information about the indicated fiducial. The position information may include, for example, the coordinates of the indicated fiducial in a three-dimensional coordinate space with the patient lying in the x-y plane.

In step 170, a determination is made as to whether information for at least two fiducials to be linked has been received. If information for at least two fiducials to be linked has not been received then the process starting at step 166 may be executed. Otherwise, in step 172, the size of at least a portion of an implant 65, for example a linking structure for linking the at least two fiducials, is calculated. The size of the linking structure preferably includes a length and an offset for the linking structure. The offset may be calculated, for example by subtracting the Z co-ordinate value of the two points. The length of the linking structure may be calculated for example by using the following formula:
Length=\sqrt{(x₂−x₁)²+(y₂−y₁)²}

Preferably, the calculations are performed with respect to the image space in order to avoid errors that may be introduced due to camera movements. If desired, the dimensions of the linking structure may be displayed on display device 12. In an exemplary embodiment, based at least in part on the dimensions of the linking structure, a link is automatically selected from a plurality of available links. Information about the selected link may then be provided to the user. Furthermore, if desired, the selected link may be displayed on display device 12. The display may illustrate the selected link coupled to the adjacent fiducials. The user may use the provided information to link the adjacent fiducials to each other.

In step 174, a determination is made as to whether there are any more fiducials to be indicated. If there are more fiducials to be indicated, then the process starting at step 166 may be executed. Otherwise, the process starting at step 154 may be executed.

In an alternative embodiment, instead of or in addition to providing the user with information on an available link, the linking structure size information may be communicated to a processor-based system that controls the operations of a tooling machine so that a linking structure of the appropriate dimensions and shape may be fabricated. The linking structure may be used to link two or more points of the spine.

A technical advantage of an exemplary embodiment of the present invention is that it guides the user through different steps of a medical procedure. Another technical advantage of an exemplary embodiment of the present invention is that it provides the user with flexibility to move from one screen to another without unnecessarily constraining the user. Another technical advantage of an exemplary embodiment of the present invention is that information about the dimensions and/or shape of a linking structure may be provided to the user so that the user does not have to determine the dimensions and/or shape by trial and error.

Embodiments of the present invention may be implemented in software, hardware, application logic or a combination of software, hardware and application logic. The software, application logic and/or hardware may reside on processor-based system 16 or on a removable storage medium. If desired, part of the software, application logic and/or hardware may reside on processor-based system 16 and part of the software, application logic and/or hardware may reside on the removable storage medium.

If desired, the different steps discussed herein may be performed in any order and/or concurrently with each other. Furthermore, if desired, one or more of the above described steps may be optional or may be combined without departing from the scope of the present invention.

While the invention has been particularly shown and described by the foregoing detailed description, it will be understood by those skilled in the art that various other changes in form and detail may be made without departing from the spirit and scope of the invention.

1. An apparatus for computer-assisted spinal fixation, comprising a storage medium for storing a spinal fixation application which, when executed by a processor, displays a series of interface images for assisting a user with a spinal fixation procedure.

2. The apparatus of claim 1, wherein said spinal fixation application is operable to cooperate with a tracking system to provide real-time pedicle entry point location assistance to said user during said spinal fixation procedure.

3. The apparatus of claim 1, wherein said spinal fixation application is operable to provide a linking structure size information to said user.

4. The apparatus of claim 1, wherein said spinal fixation application is operable to display a virtual representation of at least a portion of a spine to said user for said spinal fixation procedure.

5. The apparatus of claim 1, wherein said spinal fixation application is operable to prompt said user to select a plurality of fiducials to be used for said spinal fixation procedure.

6. The apparatus of claim 1, wherein said spinal fixation application is operable to visually indicate to said user that a requisite number of fiducials for said spinal fixation procedure have not been selected.

7. The apparatus of claim 1, wherein said spinal fixation application is operable to display to said user sizes of a plurality of implants for said spinal fixation procedure.

8. The apparatus of claim 1, wherein said spinal fixation application is operable to assist said user in determining a size of a structure for linking at least two portions of a spine.

9. A computer-assisted spinal fixation method, comprising:
receiving an input from a user; and
displaying on a display device a series of interface images for assisting said user with a spinal fixation procedure.

10. The method of claim 9, further comprising enabling said user to select a pedicle entry tool from a plurality of predetermined pedicle entry tools displayed in a predetermined one of said series of interface images.

11. The method of claim 9, further comprising enabling said user to select a pedicle reaming tool from a plurality of predetermined pedicle reaming tools displayed in a predetermined one of said series of interface images.

12. The method of claim 9, further comprising enabling said user to select an implant insertion tool from a plurality of implant insertion tools displayed in a predetermined one of said series of interface images.

13. The method of claim 9, further comprising receiving information on a plurality of fiducials from said user in response to displaying a predetermined one of said series of interface images.

14. The method of claim 9, further comprising visually indicating, on a predetermined one of said series of interface images, that a requisite number of fiducials for said spinal fixation procedure have not been selected.

15. The method of claim 9, further comprising:
calculating a tip accuracy value for at least one of a plurality of user-selected tools; and
displaying in an image of said series of interface images said calculated tip accuracy value.

16. The method of claim 9, further comprising:
calculating an axis accuracy value for at least one of a plurality of user-selected tools; and
displaying in an image of said series of interface images said calculated axis accuracy value.

17. The method of claim 9, further comprising displaying a virtual representation of at least a portion of a spine in at least one of said series of interface images.

18. The method of claim 17, further comprising highlighting in said virtual representation a plurality of fiducials previously selected by said user.

19. The method of claim 18, further comprising prompting said user to indicate said highlighted fiducials on said portion of said spine.

20. The method of claim 19, further comprising determining position information of said highlighted fiducials based at least in part on said indication by said user.

21. The method of claim 20, further comprising calculating a registration error based at least in part on said determined position information.

22. The method of claim 9, further comprising displaying, in an image of said series of interface images, size information of a plurality of implants for said spinal fixation procedure.

23. The method of claim 22, further comprising allowing said user to select an implant from said plurality of implants.

24. The method of claim 23, further comprising receiving size information for said selected implant.

25. The method of claim 17, further comprising displaying in real-time an image of a tool overlaid on said virtual representation, said image of said tool including an image of an implant selected by said user, said image of said implant extending from an end of said image of said tool.

26. The method of claim 25, wherein at least one dimension of said image of said implant is proportional to a corresponding dimension of said implant.

27. The method of claim 9, further comprising determining a size of a structure for linking at least two portions of a spine.

28. The method of claim 19, further comprising receiving position information for said highlighted fiducials.

29. The method of claim 28, further comprising calculating a size of at least a portion of a structure for linking at least two of said highlighted fiducials.

30. The method of claim 28, further comprising calculating a size of at least a portion of a structure for linking at least two of said highlighted fiducials based at least in part on position information of said at least two of said highlighted fiducials.

31. The method of claim 29, wherein said size comprises a length and an offset between two points of said at least a portion of said structure.

32. The method of claim 29, further comprising displaying said size in an image of said series of interface images.

33. The method of claim 29, further comprising automatically selecting a link from a plurality of links based at least in part on said calculated size.

34. The method of claim 33, further comprising providing information identifying said selected link to said user.

35. The method of claim 33, further comprising displaying said selected link coupled to said virtual representation of said portion of said spine.

36. The method of claim 29, further comprising providing said size to a processor-based system for controlling a tooling machine, said tooling machine fabricating said structure of appropriate size.
